(19) United States
(12) Patent Application Publication
(10) Pub. No.: US 2026/0066116 A1
(43) Pub. Date: Mar. 5, 2026

(54) INFORMATION PROCESSING APPARATUS, INFORMATION PROCESSING METHOD, AND NON-TRANSITORY COMPUTER READABLE MEDIUM

(71) Applicant: NEC Corporation, Tokyo (JP)

(72) Inventors: Makoto YASUKAWA, Tokyo (JP); Ryo TOMARU, Tokyo (JP)

(73) Assignee: NEC Corporation, Tokyo (JP)

(21) Appl. No.: 19/303,707

(22) Filed: Aug. 19, 2025

(30) Foreign Application Priority Data
Aug. 27, 2024 (JP) 2024-145689

(51) Int. Cl. G16H 50/20; A43B 3/44; A61B 5/00; A61B 5/11; G16H 10/60; G16H 40/67

(52) U.S. Cl. CPC G16H 50/20; A43B 3/44; A61B 5/112; A61B 5/6807; G16H 10/60; G16H 40/67

(57) ABSTRACT

An information processing apparatus acquires sensing data obtained by continuously sensing activities in daily life of one or a plurality of users, extract one or a plurality of target persons from the one or the plurality of users by using the acquired sensing data, and generate assessment information regarding the one or the plurality of target persons by using the sensing data regarding the extracted one or the plurality of target persons.

### INCORPORATION BY REFERENCE

This application is based upon and claims the benefit of priority from Japanese patent application No. 2024-145689, filed on Aug. 27, 2024, the disclosure of which is incorporated herein in its entirety by reference.

### TECHNICAL FIELD

The present disclosure relates to an information processing apparatus, an information processing method, and a non-transitory computer readable medium.

### BACKGROUND ART

There is known a technique of performing various estimations related to support and care for people who need assistance, nursing care, and the like or people who may need assistance, nursing care, and the like. For example, Patent Literature 1 discloses a technique of acquiring position data of a site including a toe of a subject in chronological order, extracting a feature amount based on the acquired position data, and inputting the feature amount to a learning model to estimate that the subject has frailty or mild cognitive impairment.

[Patent Literature 1]
Japanese Unexamined Patent Application Publication No. 2022-92940

### SUMMARY

As described above, the state of people who need assistance, nursing care, or the like or people who may need assistance, nursing care, or the like can greatly change according to elapse of time or change in environment. Therefore, in order to appropriately assist and perform nursing care for people as described above, it is preferable to perform continuous monitoring and intervention before the condition deteriorates.

However, in order to perform such continuous monitoring and intervention, human resources are usually required every time monitoring and intervention are performed. For example, in the technique described in JP 2022-92940 A, in order to confirm the state of the subject, it is necessary to guide the subject to an area where a camera is installed, to cause the subject to walk in the area, and to photograph such a state.

The present disclosure has been made in view of the above problems, and an example object of the present disclosure is to provide a technology capable of suitably performing assessment regarding people who need assistance, nursing care, or the like, or people who may need assistance, nursing care, or the like, while suppressing the human resources required.

An information processing apparatus according to an example aspect of the present disclosure including:
at least one memory storing instructions, and
at least one processor executing the instructions to:
acquire sensing data obtained by continuously sensing activities in daily life of one or a plurality of users,
extract one or a plurality of target persons from the one or the plurality of users by using the acquired sensing data, and
generate assessment information regarding the one or the plurality of target persons by using the sensing data regarding the extracted one or the plurality of target persons.

An information processing method according to an example aspect of the present disclosure, includes, by at least one processor,
acquiring sensing data obtained by continuously sensing activities in daily life of one or a plurality of users,
extracting one or a plurality of target persons from the one or the plurality of users by using the acquired sensing data, and
generating assessment information regarding the one or the plurality of target persons by using the sensing data regarding the extracted one or the plurality of target persons.

A non-transitory computer readable medium according to an example aspect of the present disclosure stores a program executed by a computer. The program causes the computer to execute processing of:
acquiring sensing data obtained by continuously sensing activities in daily life of one or a plurality of users,
extracting one or a plurality of target persons from the one or the plurality of users by using the acquired sensing data, and
generating assessment information regarding the one or the plurality of target persons by using the sensing data regarding the extracted one or the plurality of target persons.

According to one exemplary aspect of the present disclosure, it is possible to suitably perform assessment regarding people who need assistance, nursing care, or the like, or people who may need assistance, nursing care, or the like, while suppressing necessary human resources.

### BRIEF DESCRIPTION OF DRAWINGS

The above and other aspects, features and advantages of the present disclosure will become more apparent from the following description of certain exemplary embodiments when taken in conjunction with the accompanying drawings, in which:

FIG. 1 is a block diagram illustrating a configuration of an information processing apparatus according to the present disclosure;

FIG. 2 is a flowchart illustrating a flow of an information processing method according to the present disclosure;

FIG. 3 is a block diagram illustrating a configuration of an information processing system according to the present disclosure;

FIG. 4 is a block diagram illustrating a configuration of an information processing system according to the present disclosure;

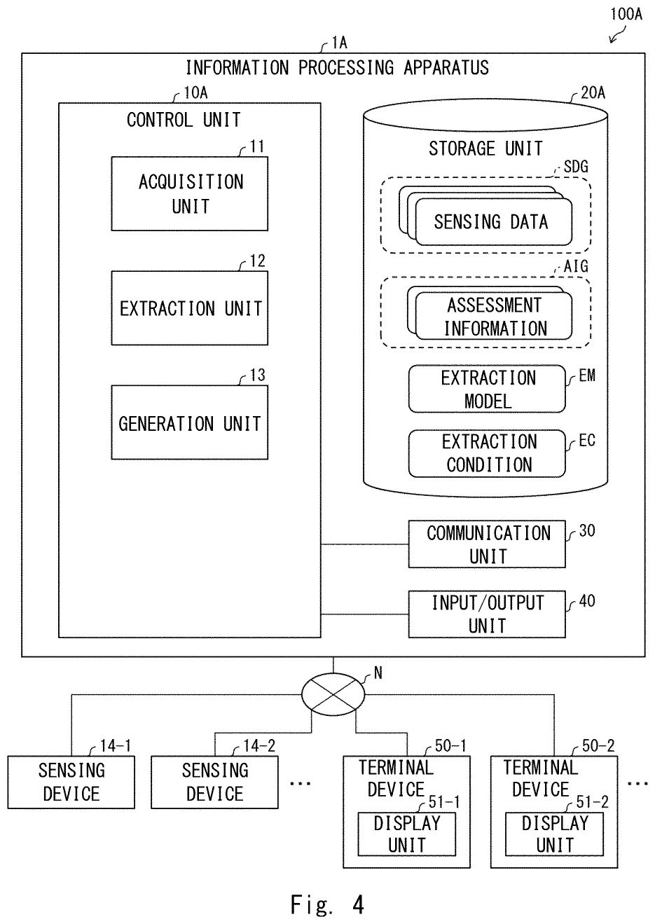

FIG. 5 is a diagram for describing processing by the information processing system according to the present disclosure;

FIG. 6 is a diagram for describing processing by the information processing system according to the present disclosure;

FIG. 7 is a diagram for describing processing by the information processing system according to the present disclosure;

FIG. 8 is a diagram for describing processing by the information processing system according to the present disclosure; and

FIG. 9 is a block diagram illustrating a configuration of a computer that functions as the information processing apparatus according to the present disclosure.

### EXAMPLE EMBODIMENTS

Hereinafter, example embodiments of the present invention will be described. However, the present invention is not limited to the exemplary example embodiments to be described below, and various modifications can be made within the scope described in the claims. For example, example embodiments obtained by appropriately combining techniques (some or all of things or methods) adopted in the following exemplary example embodiments can also be included in the scope of the present invention. Example embodiments obtained by appropriately omitting some of the techniques adopted in the following exemplary example embodiments can also be included in the scope of the present invention. Advantages mentioned in the following exemplary example embodiments are examples of advantages expected in the exemplary example embodiments, and do not define extensions of the present invention. That is, example embodiments that do not achieve the effects mentioned in the following exemplary example embodiments can also be included in the scope of the present invention.

### First Exemplary Example Embodiment

A first exemplary example embodiment that is an example of an example embodiment of the present invention will be described in detail with reference to the drawings. The present exemplary example embodiment is a basic form of each exemplary example embodiment to be described below. An application range of each technique adopted in the present exemplary example embodiment is not limited to the present exemplary example embodiment. That is, each technique adopted in the present exemplary example embodiment can also be adopted in the other exemplary example embodiments included in the present disclosure within a range in which no particular technical problem occurs. In addition, each technique illustrated in the drawings referred to for describing the present exemplary example embodiment can also be adopted in the other exemplary example embodiments included in the present disclosure within a range in which no particular technical problem occurs.

### Configuration of Information Processing Apparatus

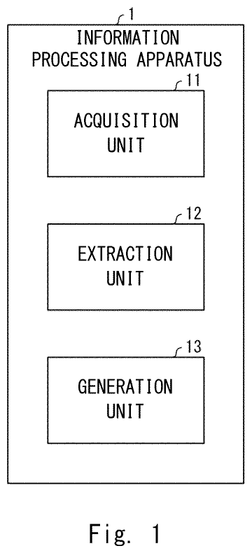

A configuration of an information processing apparatus 1 according to the present exemplary example embodiment will be described with reference to FIG. 1. The information processing apparatus 1 continuously senses activities in daily life of a user of a service provided by using the information processing apparatus 1, and generates assessment information regarding the target person extracted by using the result of the sensing. In the following description, a “user” refers to a user of a service provided using the information processing apparatus 1 as described above. The “user” is, by way of an example, people who need assistance, nursing care, or the like, or people who may need assistance, nursing care, or the like, but this does not limit the present exemplary example embodiment. In addition, the “user” may be, by way of an example, a care receiver in an elderly home, a nursing facility, or the like, but this also does not limit the present exemplary example embodiment. As illustrated in FIG. 1, the information processing apparatus 1 includes an acquisition unit 11, an extraction unit 12, and a generation unit 13.

### Acquisition Unit 11

The acquisition unit 11 acquires sensing data obtained by continuously sensing activities in daily life of one or a plurality of users. Here, the activities in daily life of the user include, as an example, movement by walking or the like, diet, dressing, excretion, bathing, dressing, and the like, but these examples do not limit the present exemplary example embodiment.

In addition, as an example, the sensing data is data obtained by continuously sensing an activity in daily life of the user by a wearable device worn by the user, but is not limited thereto. Furthermore, the sensing data may include data obtained by continuously sensing at least any of a vital sign, an activity level, a pose, and a walking feature (gait) of the user, but this also does not limit the present exemplary example embodiment. Moreover, the sensing data may include data acquired from an insole-type sensor for sensing the walking feature (gait) of the user, but this also does not limit the present exemplary example embodiment. Furthermore, the sensing data may include pose data or gait data imaged by a camera included in a terminal device such as a smartphone.

### Extraction Unit 12

The extraction unit 12 extracts one or a plurality of target persons from the one or a plurality of users using the sensing data acquired by the acquisition unit 11. The specific extraction processing by the extraction unit 12 is not limited to the present exemplary example embodiment, but as an example, the extraction unit 12 may be configured to extract the target person by determining whether the sensing data satisfies a predetermined extraction condition. Alternatively, the extraction unit 12 may be configured to extract the target person by inputting the sensing data to a learned extraction model optimized by machine learning such as deep learning. The term “target person” indicates that the target person is a target of assessment information to be described later as an example. Alternatively, the “target person” may be understood to refer to a facility user in need of individual intervention for the purpose of nursing care or falling prevention. However, these examples are not intended to limit the present exemplary example embodiment.

### Generation Unit 13

The generation unit 13 generates assessment information regarding the one or the plurality of target persons extracted by the extraction unit 12. As an example, the generation unit 13 generates assessment information regarding the target person by using the sensing data regarding the target person. Although a specific configuration of the assessment information is not limited to the present exemplary example embodiment, as an example, the assessment information may include information indicating at least either the risk of falling of the target person and the degree of frailty. In addition, the assessment information may include information indicating a risk or degree of mild cognitive impairment (MCI).

The assessment information regarding the target person generated by the generation unit 13 is presented to the target person or a provider who provides a service to a target person by way of an example. Here, the “provider who provides a service to a target person” is, for example,
a company that provides a service using the information processing apparatus 1 to the target person (or the user),
a medical worker (doctors, nurses, physical therapists, etc.) who works in a facility that provides a service using the information processing apparatus 1 to the target person (or the user),
a care worker (care workers, social workers, etc.) working in the facility, or
a family member of the target person,
but the present invention is not limited thereto. The “provider who provides a service to a target person” may be regarded as a provider who provides a nursing care service or the like to the target person. Furthermore, the term “service” may include provision of assistance or nursing care regardless of whether it is charged or free. Therefore, the expression “a provider who provides a service to a target person” may be expressed as “a person who assists or cares a target person”.

### Effects of Information Processing Apparatus

As described above, the information processing apparatus 1 adopts a configuration in which sensing data obtained by continuously sensing activities in daily life of one or a plurality of users is acquired, one or a plurality of target persons are extracted from the one or the plurality of users by using the sensing data, and assessment information regarding the one or the plurality of target persons is generated.

According to the above configuration, since the target person is extracted using the sensing data obtained by continuously sensing the activities in daily life of the user and the assessment information regarding the target person is generated, it is possible to suitably perform the assessment regarding the people who need assistance, nursing care and the like or the people who may need assistance, nursing care, and the like while suppressing the human resources that need to be involved.

### Flow of Information Processing Method

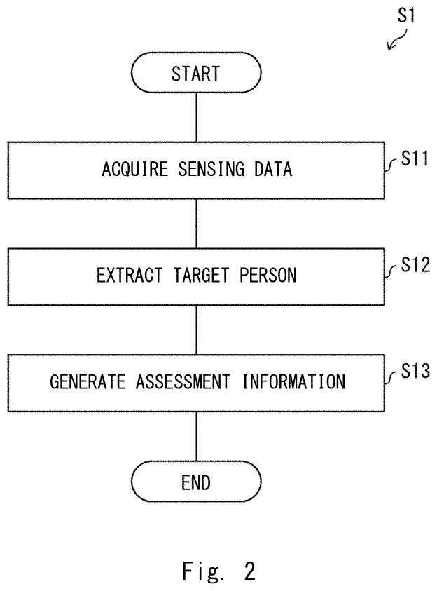

Next, a flow of an information processing method S1 according to the present exemplary example embodiment will be described with reference to FIG. 2. FIG. 2 is a flowchart illustrating the flow of the information processing method S1. As illustrated in FIG. 2, the information processing method S1 includes processing (process, step) S11 of acquiring sensing data, processing (process, step) S12 of extracting a target person, and processing (process, step) S13 of generating assessment information.

### Step S11

In step S11, the acquisition unit 11 acquires sensing data obtained by continuously sensing activities in daily life of one or a plurality of users. Since specific processing performed by the acquisition unit 11 has been described above, the description thereof will be omitted here.

### Step S12

In step S12, the extraction unit 12 extracts one or a plurality of target persons from the one or the plurality of users by using the sensing data acquired by the acquisition unit 11 in step S11. Since the specific processing by the extraction unit 12 has been described above, the description thereof will be omitted here.

### Step S13

In step S13, the generation unit 13 generates assessment information regarding the one or the plurality of target persons by using the sensing data regarding the one or the plurality of target persons extracted by the extraction unit 12 in step S12. Since specific processing performed by the generation unit 13 has been described above, the description thereof will be omitted here.

### Effects of Information Processing Method

As described above, in the information processing method S1, a configuration is adopted in which sensing data obtained by continuously sensing activities in daily life of one or a plurality of users is acquired, one or a plurality of target persons are extracted from the one or the plurality of users with reference to the sensing data, and assessment information regarding the one or the plurality of target persons is generated. With the above configuration, effects similar to those of the information processing apparatus 1 are obtained.

### Configuration of Information Processing System

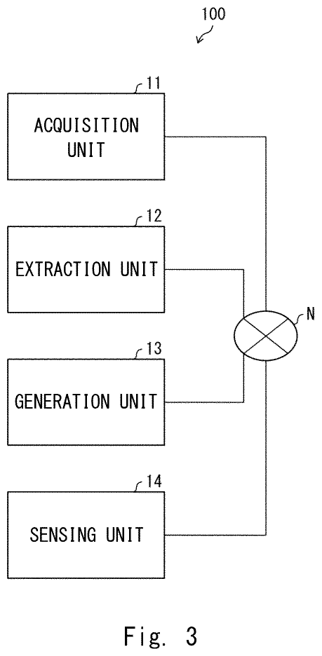

Next, a configuration of the information processing system 100 according to the present exemplary example embodiment will be described with reference to FIG. 3. As illustrated in FIG. 3, the information processing system 100 includes an acquisition unit 11, an extraction unit 12, a generation unit 13, and a sensing unit 14. Furthermore, as illustrated in FIG. 3, the acquisition unit 11, the extraction unit 12, the generation unit 13, and the sensing unit 14 are configured to be communicable via a network N. Here, the specific configuration of the network N is not limited to the present exemplary example embodiment, but as an example, a wireless local area network (LAN), a wired LAN, a wide area network (WAN), a public line network, a mobile data communication network, or a combination of these networks can be used.

### Acquisition Unit 11, Extraction Unit 12, Generation Unit 13

The acquisition unit 11, the extraction unit 12, and the generation unit 13 included in the information processing system 100 are similar to the acquisition unit 11, the extraction unit 12, and the generation unit 13 included in the information processing apparatus 1 described above, and thus description thereof is omitted here.

### Sensing Unit 14

The sensing unit 14 continuously senses activities in daily life of one or a plurality of users. Then, the sensing unit 14 supplies sensing data obtained by the continuous sensing to the acquisition unit 11. As an example, the sensing unit 14 may be configured as a wearable device worn by the user, and may continuously sense activities in daily life of the user, but this is not the sole case. In addition, as an example, the sensing unit 14 may be configured to continuously sense at least any of a vital sign, an activity level, a pose, and a walking feature of the user, but this does not limit the present exemplary example embodiment. Furthermore, as an example, the sensing unit 14 may be an insole-type sensor for sensing the walking feature of the user, but this does not limit the present exemplary example embodiment. Moreover, the sensing unit 14 may be a camera included in a terminal device such as a smartphone. The sensing unit 14 may be configured to supply pose data or gait data imaged by the camera to the acquisition unit 11 as the sensing data.

### Effects of Information Processing System

As described above, the information processing system 100 adopts a configuration including the sensing unit 14 for continuously sensing activities of one or a plurality of users in daily life, the acquisition unit 11 for acquiring the sensing data obtained by the sensing unit 14, the extraction unit 12 for extracting one or a plurality of target persons from the one or the plurality of users by using the sensing data, and the generation unit 13 for generating assessment information regarding the one or the plurality of target persons. With the above configuration, effects similar to those of the information processing apparatus 1 are obtained.

### Second Exemplary Example Embodiment

A second exemplary example embodiment that is an example of an example embodiment of the present invention will be described in detail with reference to the drawings. Constituents that have the same functions as the constituents described in the above-described exemplary example embodiment are denoted by the same reference numerals, and the description of the constituents will be appropriately omitted. An application range of each technique adopted in the present exemplary example embodiment is not limited to the present exemplary example embodiment. That is, each technique adopted in the present exemplary example embodiment can also be adopted in the other exemplary example embodiments included in the present disclosure within a range in which no particular technical problem occurs. Each technique illustrated in each of the drawings referred to for describing the present exemplary example embodiment can be employed in the other exemplary example embodiments included in the present disclosure within a range in which no particular technical problem occurs.

### Configuration of Information Processing System

A configuration of an information processing system 100A including an information processing apparatus 1A according to the present exemplary example embodiment will be described with reference to FIG. 3. FIG. 3 is a block diagram illustrating a configuration of the information processing system 100A. As illustrated in FIG. 3, the information processing system 100A includes the information processing apparatus 1A, one or a plurality of sensing devices 14, and one or a plurality of terminal devices 50. Here, the information processing apparatus 1A, the one or the plurality of sensing devices 14, and the one or the plurality of terminal devices 50 are configured to be communicable via the network N, as illustrated in FIG. 3. Here, the specific configuration of the network N is not limited to the present exemplary example embodiment, but as an example, a wireless LAN, a wired LAN, a WAN, a public network, a mobile data communication network, near field communication, or a combination of these networks can be used.

In a case where the information processing system 100A includes a plurality of sensing devices 14, as illustrated in FIG. 3, each sensing device may be denoted with a branch number such as 14-1 and 14-2. Similarly, in a case where the information processing system 100A includes a plurality of terminal devices 50, as illustrated in FIG. 3, each terminal device may be denoted with a branch number such as 50-1 and 50-2.

### Sensing Device 14

The sensing device 14 continuously senses activities in daily life of the user of the information processing system 100A. Then, the sensing device 14 supplies the sensing data obtained by the continuous sensing to the information processing apparatus 1A. As an example, the sensing device 14 may be configured as a wearable device worn by the user, and may continuously sense activities in daily life of the user, but this is not the sole case. In addition, as an example, the sensing device 14 may be configured to continuously sense at least any of a vital sign, an activity level, a pose, and a walking feature of the user, but this does not limit the present exemplary example embodiment. Furthermore, as an example, the sensing device 14 may be an insole-type sensor for sensing the walking feature of the user, but this does not limit the present exemplary example embodiment. Moreover, the sensing unit 14 may be a camera included in a terminal device such as a smartphone. As an example, the sensing unit 14 may be configured as a part of the terminal device 50. The sensing unit 14 may be configured to supply pose data or gait data imaged by the camera to the acquisition unit 11 as the sensing data.

In a case where there are a plurality of users of the information processing system 100A, as an example, the sensing device 14 is individually provided to each user. For example, the sensing device 14-1 is provided to the user U1, and the sensing device 14-2 is provided to the user U2.

Furthermore, in a case where the sensing device 14 is configured as a wearable device such as a wristband-type sensor or an insole-type sensor, the sensing device 14 includes a battery for supplying power to the sensor.

Furthermore, the sensing device 14 may include a power generation mechanism for generating power by activities in daily life of the user. As an example, in a case where the sensing device 14 is configured as a wristband-type sensor, the sensing device 14 may be configured to include a power generation mechanism for generating power by movement of an arm of the user. Furthermore, in a case where the sensing device 14 is configured as an insole-type sensor, the sensing device 14 may be configured to include a power generation mechanism for generating power by movement of a foot of the user. If the sensing device 14 includes the power generation mechanism, the trouble of maintenance such as charging of the sensing device 14 can be reduced, and thus the activity in daily life of the user can be suitably and continuously sensed.

### Terminal Device 50

The terminal device 50 includes a display unit 51, and displays assessment information generated by a generation unit 13 of the information processing apparatus 1A described later. In a case where there are a plurality of users of the information processing system 100A, as an example, the terminal device 50 may be individually provided to each user. For example, the terminal device 50-1 to be used by the user U1 may be provided to the user U1, and the terminal device 50-2 to be used by the user U2 may be provided to the user U2. Furthermore, the terminal device 50 may have a function as a relay device for supplying the sensing data by the sensing device 14 to the information processing apparatus 1A. For example, the terminal device 50-1 may receive the sensing data by the sensing device 14-1 that continuously senses activities in daily life of the user U1 by near field communication, and the terminal device 50-1 may supply the received sensing data to the information processing apparatus 1A via the wireless LAN.

Furthermore, the one or the plurality of terminal devices 50 may include a terminal device used by a provider who provides a service to the user. Here, the “provider who provides a service to the user” is, for example, a company, a facility, an employee working in the facility, a family member of the user, or the like that provides the service using the information processing apparatus 1A to the user, but is not limited thereto. The “provider who provides a service to the user” may be regarded as a provider who provides a nursing care service or the like to the user. Furthermore, the term “service” may include provision of assistance or nursing care regardless of whether it is charged or free. Therefore, the expression “a provider who provides a service to a user” may be expressed as “a person who assists or cares a user”.

### Information Processing Apparatus 1A

Next, a configuration of the information processing apparatus 1A will be described. As illustrated in FIG. 3, the information processing apparatus 1A includes a control unit 10A, a storage unit 20A, a communication unit 30, and an input/output unit 40.

### Communication Unit 30

The communication unit 30 communicates with an external device of the information processing apparatus 1A via the network N described above. The communication unit 30 transmits data supplied from the control unit 10A to another device, and supplies data received from another device to the control unit 10A. As an example, the communication unit 30 receives sensing data from the sensing device 14 and supplies the received sensing data to the control unit 10A or the storage unit 20A. Furthermore, the communication unit 30 transmits the assessment information generated by the generation unit 13 to the terminal device 50.

### Input/output Unit 40

Input/output devices such as a keyboard, a mouse, a display, a printer, and a touch panel are connected to the input/output unit 40. The input/output unit 40 accepts inputs of various types of information with respect to the information processing apparatus 1A from the connected input device. In addition, the input/output unit 40 outputs various types of information to a connected output device under the control of the control unit 10A. Examples of the input/output unit 40 include an interface such as, for example, a universal serial bus (USB).

### Storage Unit 20A

The storage unit 20A stores various types of data and programs used by the control unit 10A and various types of data derived by the control unit 10A. As an example, in the storage unit 20A,
sensing data group SDG
assessment information group AIG
extraction model EM
extraction condition EC
and the like are stored.

Here, the sensing data group SDG includes sensing data obtained by continuously sensing activities in daily life of one or a plurality of users by one or a plurality of sensing devices 14. In addition, the assessment information group AIG is assessment information generated by the generation unit 13 to be described later and includes assessment information regarding each of one or a plurality of target persons. The extraction model EM and the extraction condition EC are used by the extraction unit 12 described later to extract the target person. Details of the extraction model EM and the extraction condition EC will be described later.

### Control Unit 10A

As illustrated in FIG. 3, the control unit 10A includes the acquisition unit 11, the extraction unit 12, and the generation unit 13.

### Acquisition Unit 11

The acquisition unit 11 acquires sensing data obtained by continuously sensing activities in daily life of one or a plurality of users. As an example, the acquisition unit 11 acquires sensing data obtained by the sensing from each of the sensing devices 14-1 and 14-2 that continuously sense activities in daily life of the users U1, U2, . . . . Here, the activities in daily life of the user include, as an example, movement by walking or the like, eating, dressing, excretion, bathing, and dressing, as in the first exemplary example embodiment, but these examples do not limit the present exemplary example embodiment. Details of the sensing data have been described above, and thus description thereof will be omitted here.

### Extraction Unit 12

The extraction unit 12 extracts one or a plurality of target persons from the one or a plurality of users using the sensing data acquired by the acquisition unit 11. Here, as in the first exemplary example embodiment, the term “target person” indicates that the target person is a target of assessment information to be described later as an example. Alternatively, the “target person” may be understood to refer to a facility user in need of individual intervention for the purpose of nursing care or falling prevention. However, these examples are not intended to limit the present exemplary example embodiment.

The specific extraction processing by the extraction unit 12 is not limited to the present exemplary example embodiment, but as an example, the extraction unit 12 may be configured to extract the target person by determining whether the sensing data satisfies the extraction condition EC. Here, the extraction condition EC is a condition used to extract the target person, and is configured to include, as an example, a condition satisfied by the target person. As an example, the extraction condition EC is set by the extraction unit 12 based on at least either:
an average value of predetermined indexes derived from sensing data, the average value of the indexes being related to a plurality of users, and
a variance of the predetermined indexes derived from sensing data, the variance of the indexes being related to a plurality of users.

Here, as an example, the extraction condition EC may be set for each nursing home or each nursing facility.

Furthermore, as another example, the extraction unit 12 may be configured to extract the target person by inputting the sensing data to the extraction model EM optimized by machine learning such as deep learning. Here, the extraction model EM is a learned extraction model used to extract the target person, and as an example, is a model that outputs information regarding whether to be set as the target person if the sensing data is input. As an example, the extraction model EM is optimized by learning using learning data including a plurality of sets of sensing data and correct answer label regarding whether to be set as the target person. A specific configuration of the extraction model EM is not limited to the present exemplary example embodiment, but includes, as an example, a convolutional neural network (CNN), a recurrent neural network (RNN), a decision tree (Decision Tree), or the like. However, these examples are not intended to limit the present exemplary example embodiment. Here, as an example, the extraction model EM may be set for each nursing home or each nursing facility.

The extraction processing of the target person by the extraction unit 12 is not limited to the above example. As an example, the extraction unit 12 may be configured to extract the target person by further using information other than the sensing data of the user. For example, the extraction unit 12 may extract candidates of one or a plurality of target persons from a plurality of users by using age, gender, work history, information input by the worker at the previous assessment, and the like of the user, and extract the target person by the extraction condition EC or the extraction model EM using the extracted candidates of the one or the plurality of target persons as a population. Furthermore, the extraction unit 12 may extract the target person by using past sensing data of the user other than the latest sensing data regarding the user. As an example, in a case where a difference between an index derived from the past sensing data of a certain user and an index derived from the latest sensing data of the user is equal to or larger than a predetermined magnitude, the user may be extracted as the target person.

### Generation Unit 13

The generation unit 13 generates assessment information regarding the one or the plurality of target persons extracted by the extraction unit 12. As an example, the generation unit 13 generates assessment information regarding the target person by using the sensing data regarding the target person. Although a specific configuration of the assessment information is not limited to the present exemplary example embodiment, as an example, the assessment information may include information indicating at least either the risk of falling of the target person and the degree of frailty. In addition, the assessment information may include information indicating a risk or degree of mild cognitive impairment (MCI).

The assessment information regarding the target person generated by the generation unit 13 is presented to the target person or a provider who provides a service to a target person by way of an example. Here, the “provider who provides a service to a target person” is, as an example, similarly to the first exemplary example embodiment,
a company that provides a service using the information processing apparatus 1 to the target person (or the user),
a medical worker (doctors, nurses, physical therapists, etc.) who works in a facility that provides a service using the information processing apparatus 1 to the target person (or the user),
a care worker (care workers, social workers, etc.) working in the facility, or
a family member of the target person,
but the present invention is not limited thereto. In addition, similarly to the first exemplary example embodiment, the “provider who provides a service to the target person” may be regarded as a provider who provides nursing care services or the like to the target person. Furthermore, the term “service” may include provision of assistance or nursing care regardless of whether it is charged or free. Therefore, the expression “a provider who provides a service to a target person” may be expressed as “a person who assists or cares a target person”. A specific example of the assessment information will be described later.

### First Processing Example

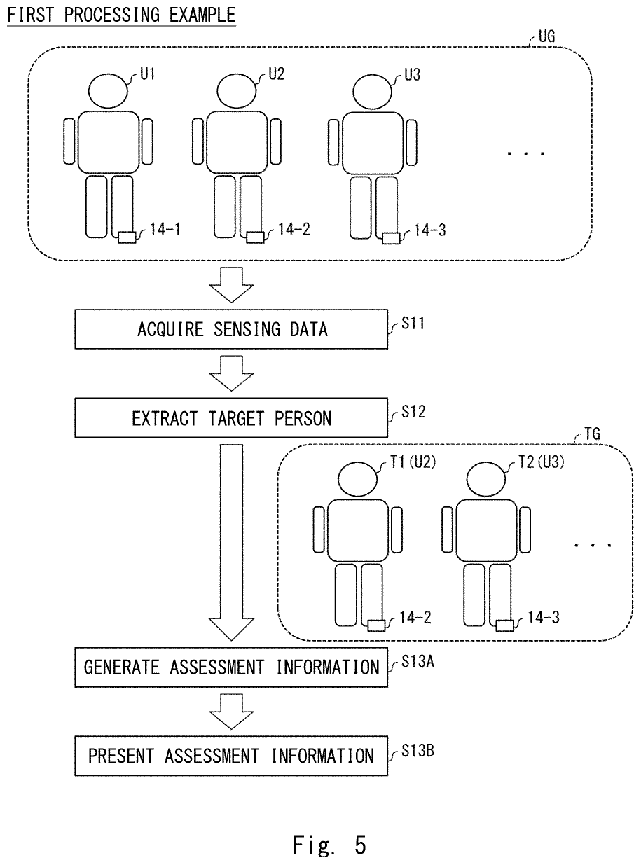

FIG. 5 is a diagram schematically illustrating a flow of processing by the information processing system 100A. As illustrated in FIG. 5, as an example, a plurality of users U1, U2, U3, . . . wear sensing devices 14-1, 14-2, 14-3, respectively, and the sensing devices 14-1, 14-2, 14-3 continuously sense activities in daily life of each of the users U1, U2, U3, . . . , and the acquisition unit 11 acquires sensing data obtained by the sensing (S11 in FIG. 5). In the example illustrated in FIG. 5, each of the plurality of users U1, U2, U3, . . . wears an insole-type sensor that senses the walking feature (gait) of the user as the sensing devices 14-1, 14-2, and 14-3, but this does not limit the present example.

Subsequently, the extraction unit 12 extracts one or a plurality of target persons from the one or the plurality of users using the sensing data acquired by the acquisition unit 11 (S12 in FIG. 5). In the example illustrated in FIG. 5, a plurality of target persons T1, T2, . . . are extracted from a plurality of users U1, U2, U3, . . . by the extraction processing by the extraction unit 12. Here, in the example illustrated in FIG. 5, the target person T1 is the user U2 and the target person T2 is the user U3, but this does not limit the present example.

Subsequently, the generation unit 13 generates assessment information regarding the one or the plurality of target persons extracted by the extraction unit 12 (S13A in FIG. 5). Then, the generation unit 13 presents the generated assessment information (S13A in FIG. 5).

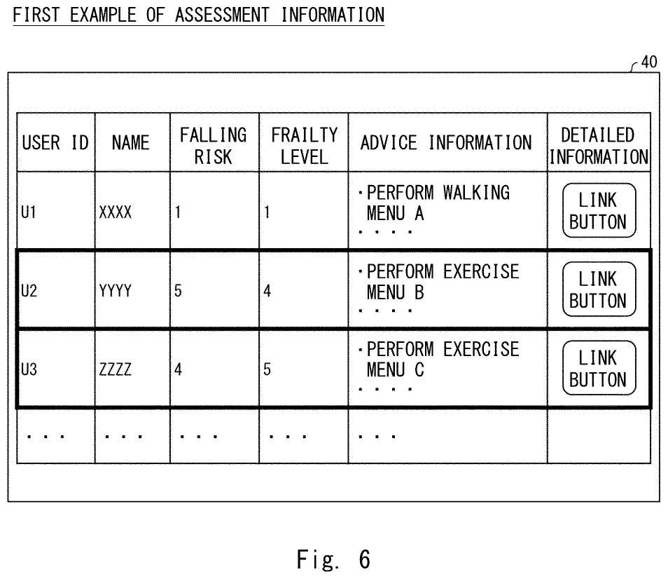

FIG. 6 illustrates an example of the assessment information generated by the generation unit 13. As an example, the assessment information is information generated by the generation unit 13 for “a provider who provides a service to the target person” and displayed via the input/output unit 40 of the information processing apparatus 1A. As illustrated in FIG. 6, the assessment information includes,
user ID
name
falling risk
frailty level
advice information
link to detailed information
and the like relating to a plurality of users (target persons). More specifically, the assessment information illustrated in FIG. 6 includes various types of information relating to the target person T1 (U2) and various types of information relating to the target person T2 (U3). Furthermore, in the example illustrated in FIG. 6, various types of information regarding the target person are displayed by emphasized display such as a frame box in such a way as to be distinguishable from other users not extracted as the target person. The “provider who provides a service to the target person” can suitably execute the assessment (monitoring, counseling, intervention, etc.) on each target person by referring to the assessment information.

The falling risk and the frailty level included in the assessment information are derived by the generation unit 13 or the extraction unit 12 based on the sensing data relating to the target person as an example. Furthermore, as an example, the advice information included in the assessment information is selected by the generation unit 13 or the extraction unit 12 from a plurality of candidates of the advice information based on the sensing data relating to the target person.

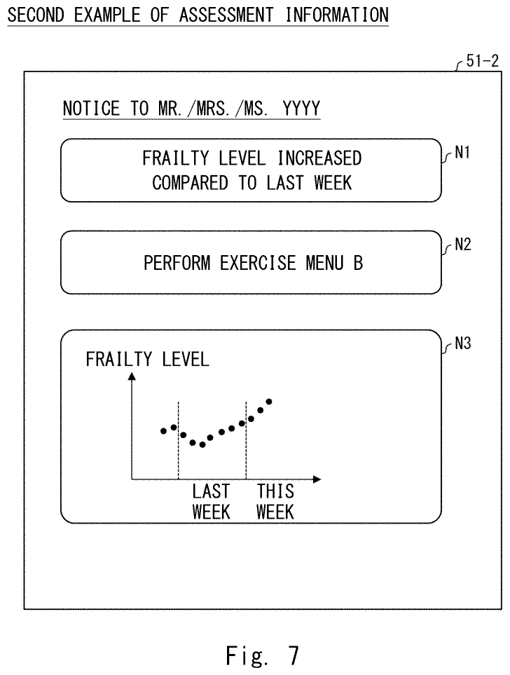

FIG. 7 illustrates another example of the assessment information generated by the generation unit 13. The assessment information is, by way of example, information generated by the generation unit 13 for the “target person” and displayed via the display unit (the display unit 51-2 in the example of FIG. 7) of the terminal device 50 used by the target person. As an example, as illustrated in FIG. 7, the assessment information includes,
notification information N1 regarding the falling risk or the frailty level of the target person
advice information N2 to the target person
past history N3 regarding the falling risk or the frailty level.

The user can suitably recognize his/her state and what to do by referring to the assessment information.

Although FIG. 7 illustrates the assessment information for the target person extracted by the extraction unit 12, the generation unit 13 may also create assessment information for the user not extracted as the target person. As an example, the generation unit 13 causes the display unit of the terminal device 50-1 used by the user U1 illustrated in FIG. 6 to display the assessment information including,
the fact that the falling risk and the frailty level both have low values,
the fact that performing a walking menu A is recommended,
past history regarding the falling risk and the flail level.

As described above, the assessment information generated by the generation unit 13 may include information that assists decision making of the user or a provider who provides a service to the user.

### Second Processing Example

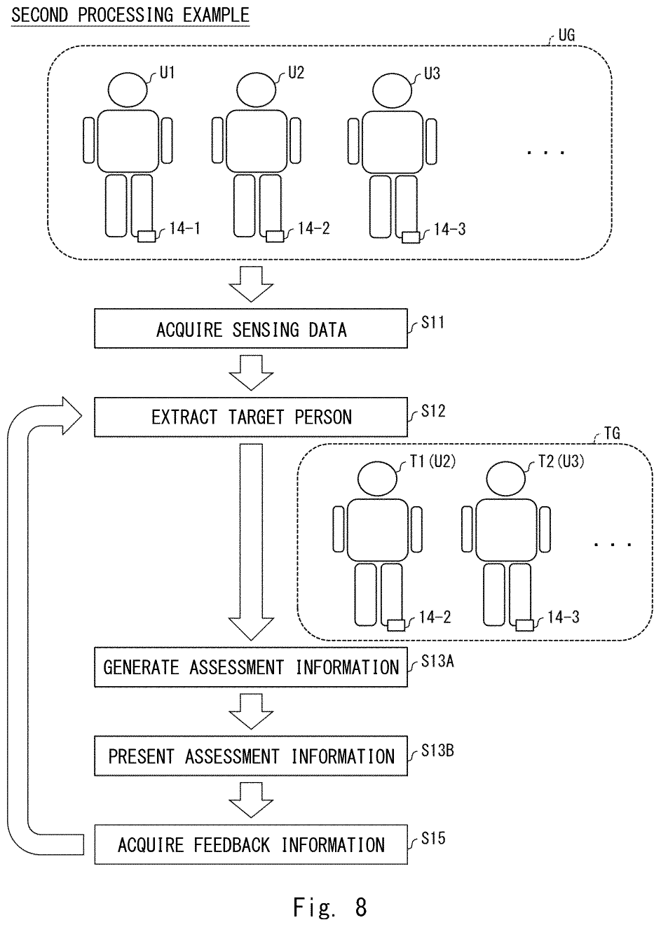

Next, another example of the flow of processing by the information processing system 100A will be described with reference to FIG. 8. FIG. 8 is a diagram schematically illustrating another example of the flow of processing by the information processing system 100A. The present processing example illustrated in FIG. 8 is different from the first processing example illustrated in FIG. 5 in the following points.

That is, in the present processing example, after presenting the assessment information, the information processing system 100A may further acquire feedback information from
a user to whom the assessment information is provided, or
a provider who is provided with the assessment information and who provides a service to the user (S15 in FIG. 8). As an example, the information processing system 100A may acquire feedback information from the user to whom the assessment information is provided via the terminal device 50. Furthermore, the information processing system 100A may acquire feedback information indicating feedback from the provider via the input/output unit 40.

Furthermore, in the present processing example, as illustrated in FIG. 8, these pieces of feedback information are used in the next target person extraction processing (S12 in FIG. 8). As an example, the extraction unit 12 may be configured to update the extraction model EM or the extraction condition EC by using the feedback information. As an example, in a case where the feedback information regarding a certain subject indicates that both the falling risk and the frailty level of the target person are low, the extraction unit 12 may be configured to update the extraction model EM or the extraction condition EC in such a way that the target person is less likely to be extracted as the target person in the next extraction processing.

### Effects of Information Processing System 100A

As described above, the information processing system 100A adopts a configuration including the sensing unit 14 for continuously sensing activities in daily life of one or a plurality of users, the acquisition unit 11 for acquiring the sensing data obtained by the sensing unit 14, the extraction unit 12 for extracting one or a plurality of target persons from the one or the plurality of users by using the sensing data, and the generation unit 13 for generating assessment information regarding the one or the plurality of target persons.

According to the above configuration, since the target person is extracted using the sensing data obtained by continuously sensing the activities in daily life of the user and the assessment information regarding the target person is generated, it is possible to suitably perform the assessment regarding the people who need assistance, nursing care and the like or the people who may need assistance, nursing care, and the like while suppressing the human resources that need to be involved.

Furthermore, as described above, by referring to the assessment information for the service provider generated by the generation unit 13, the service provider can suitably execute assessment (monitoring, counseling, intervention, etc.) on each target person.

Furthermore, as described above, by referring to the assessment information for the target person generated by the generation unit 13, the target person can suitably recognize his/her state and what to do.

### Implementation Example by Software

Some or all of the functions of the information processing apparatuses 1 and 1A and the terminal device 50 (hereinafter, also referred to as “each of the above devices”) may be implemented by hardware such as an integrated circuit (IC chip) or may be implemented by software.

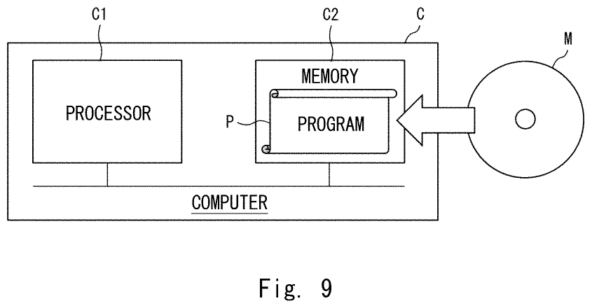

In the latter case, each of the above apparatuses is implemented by, for example, a computer that executes a command of a program which is software for implementing each function. FIG. 9 illustrates an example of such a computer (hereinafter, referred to as a computer C). FIG. 9 is a block diagram illustrating a hardware configuration of a computer C functioning as each of the above devices.

The computer C includes at least one processor C1 and at least one memory C2. A program P causing the computer C to operate as each of the above apparatuses is recorded in the memory C2. In the computer C, the processor C1 reads the program P from the memory C2 and executes the program P to implement each function of each of the above apparatuses.

As the processor C1, for example, a central processing unit (CPU), a graphic processing unit (GPU), a digital signal processor (DSP), a micro processing unit (MPU), a floating point number processing unit (FPU), a physics processing unit (PPU), a tensor processing unit (TPU), a quantum processor, a microcontroller, or a combination thereof can be used. As the memory C2, for example, a flash memory, a hard disk drive (HDD), a solid state drive (SSD), or a combination thereof can be used.

The computer C may further include a random access memory (RAM) for developing the program P at the time of execution and temporarily storing various types of data. In addition, the computer C may further include a communication interface for transmitting and receiving data to and from other apparatuses. The computer C may further include an input/output interface for connecting input/output devices such as a keyboard, a mouse, a display, and a printer.

In addition, the program P can be recorded in a non-transitory tangible recording medium M readable by the computer C. As such a recording medium M, for example, a tape, a disk, a card, a semiconductor memory, a programmable logic circuit, or the like can be used. The computer C can acquire the program P via such a recording medium M. In addition, the program P can be transmitted via a transmission medium. As such a transmission medium, for example, a communication network, a broadcast wave, or the like can be used. The computer C can also acquire the program P via such a transmission medium.

A (The) program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

Each of the above functions of each of the above apparatuses may be implemented by one processor provided in one computer, may be implemented in cooperation with a plurality of processors provided in one computer, or may be implemented in cooperation with a plurality of processors provided in a plurality of computers, respectively. The program causing each of the above apparatuses to implement each of the above functions may be stored in one memory provided in one computer, may be stored in a distributed manner in a plurality of memories provided in one computer, or may be stored in a distributed manner in a plurality of memories provided in a plurality of computers, respectively.

The present disclosure includes techniques described in the following supplementary notes. However, the present invention is not limited to the techniques described in the following supplementary note, and various modifications can be made within the scope described in the claims.

### Supplementary Note 1

An information processing apparatus including:
at least one memory storing instructions, and
at least one processor executing the instructions to:
acquire sensing data obtained by continuously sensing activities in daily life of one or a plurality of users,
extract one or a plurality of target persons from the one or the plurality of users by using the acquired sensing data, and
generate assessment information regarding the one or the plurality of target persons by using the sensing data regarding the extracted one or the plurality of target persons.

### Supplementary Note 2

The information processing apparatus according to supplementary note 1, in which the sensing data includes data obtained by continuously sensing at least any of a vital sign, an activity level, a pose, and a walking feature of the user.

### Supplementary Note 3

The information processing apparatus according to supplementary note 2, in which the sensing data includes data acquired from an insole-type sensor for sensing a walking feature of the user.

### Supplementary Note 4

The information processing apparatus according to supplementary note 3, in which the assessment information includes information indicating at least either a risk of falling and a degree of frailty of the target person.

### Supplementary Note 5

The information processing apparatus according to supplementary note 1, in which the at least one processor further executes the instructions to: extract the one or the plurality of target persons from the one or the plurality of users by, determining whether the sensing data satisfies an extraction condition, or inputting the sensing data to an extraction model optimized by machine learning.

### Supplementary Note 6

The information processing apparatus according to supplementary note 5, in which the at least one processor further executes the instructions to: further acquire feedback information indicating feedback from a user to whom the assessment information is provided or a provider to whom the assessment information is provided, the provider providing a service to the user, and update the extraction model or the extraction condition by using the feedback information.

### Supplementary Note 7

The information processing apparatus according to supplementary note 1, in which the assessment information includes information that assists decision making of the user or a provider who provides a service to the user.

### Supplementary Note 8

An information processing method including,
by at least one processor,
acquiring sensing data obtained by continuously sensing activities in daily life of one or a plurality of users,
extracting one or a plurality of target persons from the one or the plurality of users by using the acquired sensing data, and
generating assessment information regarding the one or the plurality of target persons by using the sensing data regarding the extracted one or the plurality of target persons.

### Supplementary Note 9

The information processing method according to supplementary note 8, in which the sensing data includes data obtained by continuously sensing at least any of a vital sign, an activity level, a pose, and a walking feature of the user.

### Supplementary Note 10

The information processing method according to supplementary note 9, in which the sensing data includes data acquired from an insole-type sensor for sensing a walking feature of the user.

### Supplementary Note 11

The information processing method according to supplementary note 10, in which the assessment information includes information indicating at least either a risk of falling and a degree of frailty of the target person.

### Supplementary Note 12

The information processing method according to supplementary note 8, in which the at least one processor further: extracts the one or the plurality of target persons from the one or the plurality of users by, determining whether the sensing data satisfies an extraction condition, or inputting the sensing data to an extraction model optimized by machine learning.

### Supplementary Note 13

The information processing method according to supplementary note 12, in which the at least one processor further: acquires feedback information indicating feedback from a user to whom the assessment information is provided or a provider to whom the assessment information is provided, the provider providing a service to the user, and updates the extraction model or the extraction condition by using the feedback information.

### Supplementary Note 14

The information processing method according to supplementary note 8, in which the assessment information includes information that assists decision making of the user or a provider who provides a service to the user.

### Supplementary Note 15

A non-transitory computer readable medium storing a program executed by a computer, the program causing the computer to execute processing of:
acquiring sensing data obtained by continuously sensing activities in daily life of one or a plurality of users,
extracting one or a plurality of target persons from the one or the plurality of users by using the acquired sensing data, and
generating assessment information regarding the one or the plurality of target persons by using the sensing data regarding the extracted one or the plurality of target persons.

### Supplementary Note 16

The non-transitory computer readable medium according to supplementary note 15, in which the sensing data includes data obtained by continuously sensing at least any of a vital sign, an activity level, a pose, and a walking feature of the user.

### Supplementary Note 17

The non-transitory computer readable medium according to supplementary note 16, in which the sensing data includes data acquired from an insole-type sensor for sensing a walking feature of the user.

### Supplementary Note 18

The non-transitory computer readable medium according to supplementary note 17, in which the assessment information includes information indicating at least either a risk of falling and a degree of frailty of the target person.

### Supplementary Note 19

The non-transitory computer readable medium according to supplementary note 15, in which the program further causes the computer to execute processing of: extracting the one or the plurality of target persons from the one or the plurality of users by, determining whether the sensing data satisfies an extraction condition, or inputting the sensing data to an extraction model optimized by machine learning.

### Supplementary Note 20

The non-transitory computer readable medium according to supplementary note 19, in which the program further causes the computer to execute processing of: further acquiring feedback information indicating feedback from a user to whom the assessment information is provided or a provider to whom the assessment information is provided, the provider providing a service to the user, and updating the extraction model or the extraction condition by using the feedback information.

### Supplementary Note 21

The non-transitory computer readable medium according to supplementary note 15, in which the assessment information includes information that assists decision making of the user or a provider who provides a service to the user.

### Supplementary Note 22

An information processing system including:
at least one memory storing instructions, and
at least one processor executing the instructions to:
acquire sensing data obtained by continuously sensing activities in daily life of one or a plurality of users,
extract one or a plurality of target persons from the one or the plurality of users by using the acquired sensing data, and
generate assessment information regarding the one or the plurality of target persons by using the sensing data regarding the extracted one or the plurality of target persons.

Some or all of the elements described in supplementary notes 2 to 7 subordinate to supplementary note 1 can also be subordinate to supplementary note 22 by the same subordinate relationship as supplementary notes 2 to 7. Some or all of the elements described in any supplementary note may be applied to various types of hardware, software, recording means for recording software, systems, and methods.

While the present disclosure has been particularly shown and described with reference to example embodiments thereof, the present disclosure is not limited to these example embodiments. It will be understood by those of ordinary skill in the art that various changes in form and details may be made therein without departing from the sprit and scope of the present disclosure as defined by the claims. And each embodiment can be appropriately combined with at least one of embodiments.

Each of the drawings or figures is merely an example to illustrate one or more example embodiments. Each figure may not be associated with only one particular example embodiment, but may be associated with one or more other example embodiments. As those of ordinary skill in the art will understand, various features or steps described with reference to any one of the figures can be combined with features or steps illustrated in one or more other figures, for example to produce example embodiments that are not explicitly illustrated or described. Not all of the features or steps illustrated in any one of the figures to describe an example embodiment are necessarily essential, and some features or steps may be omitted. The order of the steps described in any of the figures may be changed as appropriate.

What is claimed is:

1. An information processing apparatus comprising:
at least one memory storing instructions, and
at least one processor executing the instructions to:
acquire sensing data obtained by continuously sensing activities in daily life of one or a plurality of users;
extract one or a plurality of target persons from the one or the plurality of users by using the acquired sensing data, and
generate assessment information regarding the one or the plurality of target persons by using the sensing data regarding the extracted one or the plurality of target persons.

2. The information processing apparatus according to claim 1, wherein the sensing data includes data obtained by continuously sensing at least any of a vital sign, an activity level, a pose, and a walking feature of the user.

3. The information processing apparatus according to claim 2, wherein the sensing data includes data acquired from an insole-type sensor for sensing a walking feature of the user.

4. The information processing apparatus according to claim 3, wherein the assessment information includes information indicating at least either a risk of falling and a degree of frailty of the target person.

5. The information processing apparatus according to claim 1, wherein the at least one processor further executes the instructions to: extract the one or the plurality of target persons from the one or the plurality of users by, determining whether the sensing data satisfies an extraction condition, or inputting the sensing data to an extraction model optimized by machine learning.

6. The information processing apparatus according to claim 5, wherein the at least one processor further executes the instructions to: further acquire feedback information indicating feedback from a user to whom the assessment information is provided or a provider to whom the assessment information is provided, the provider providing a service to the user, and update the extraction model or the extraction condition by using the feedback information.

7. The information processing apparatus according to claim 1, wherein the assessment information includes information that assists decision making of the user or a provider who provides a service to the user.

8. An information processing method comprising:
by at least one processor,
acquiring sensing data obtained by continuously sensing activities in daily life of one or a plurality of users;
extracting one or a plurality of target persons from the one or the plurality of users by using the acquired sensing data; and
generating assessment information regarding the one or the plurality of target persons by using the sensing data regarding the extracted one or the plurality of target persons.

9. The information processing method according to claim 8, wherein the sensing data includes data obtained by continuously sensing at least any of a vital sign, an activity level, a pose, and a walking feature of the user.

10. The information processing method according to claim 9, wherein the sensing data includes data acquired from an insole-type sensor for sensing a walking feature of the user.

11. The information processing method according to claim 10, wherein the assessment information includes information indicating at least either a risk of falling and a degree of frailty of the target person.

12. The information processing method according to claim 8, wherein the at least one processor further: extracts the one or the plurality of target persons from the one or the plurality of users by, determining whether the sensing data satisfies an extraction condition, or inputting the sensing data to an extraction model optimized by machine learning.

13. The information processing method according to claim 12, wherein the at least one processor further: acquires feedback information indicating feedback from a user to whom the assessment information is provided or a provider to whom the assessment information is provided, the provider providing a service to the user; and updates the extraction model or the extraction condition by using the feedback information.

14. The information processing method according to claim 8, wherein the assessment information includes information that assists decision making of the user or a provider who provides a service to the user.

15. A non-transitory computer readable medium storing a program executed by a computer, the program causing the computer to execute processing of:
acquiring sensing data obtained by continuously sensing activities in daily life of one or a plurality of users;
extracting one or a plurality of target persons from the one or the plurality of users by using the acquired sensing data; and
generating assessment information regarding the one or the plurality of target persons by using the sensing data regarding the extracted one or the plurality of target persons.

16. The non-transitory computer readable medium according to claim 15, wherein the sensing data includes data obtained by continuously sensing at least any of a vital sign, an activity level, a pose, and a walking feature of the user.

17. The non-transitory computer readable medium according to claim 16, wherein the sensing data includes data acquired from an insole-type sensor for sensing a walking feature of the user.

18. The non-transitory computer readable medium according to claim 17, wherein the assessment information includes information indicating at least either a risk of falling and a degree of frailty of the target person.

19. The non-transitory computer readable medium according to claim 15, wherein the program further causes the computer to execute processing of: extracting the one or the plurality of target persons from the one or the plurality of users by, determining whether the sensing data satisfies an extraction condition, or inputting the sensing data to an extraction model optimized by machine learning.

20. The non-transitory computer readable medium according to claim 19, wherein the program further causes the computer to execute processing of: further acquiring feedback information indicating feedback from a user to whom the assessment information is provided or a provider to whom the assessment information is provided, the provider providing a service to the user; and updating the extraction model or the extraction condition by using the feedback information.
